(19) United States
(12) Patent Application Publication
(10) Pub. No.: US 2003/0224421 A1
(43) Pub. Date: Dec. 4, 2003

(54) New oxazine dyes and their use as fluorescent markers

(76) Inventors: Rupert Herrmann, Weilheim (DE); Hans-Peter Josel, Weilheim (DE); Karl-Heinz Drexhage, Siegen (DE); Nicolaas-Joseph Marx, Kirchhundem (NL)

Correspondence Address: FULBRIGHT & JAWORSKI, LLP, 666 FIFTH AVE, NEW YORK, NY, 10103-3198, US

(21) Appl. No.: 10/407,768

(22) Filed: Apr. 3, 2003

Related U.S. Application Data
(62) Division of application No. 09/141,950, filed on Aug. 28, 1998.
Application No. 09/141,950 is a division of application No. 08/662,713, filed on Jun. 10, 1996, now abandoned.

(30) Foreign Application Priority Data
Jun. 10, 1995 (DE) 195 21 231.2

(51) Int. Cl. C12Q 1/68; G01N 33/53; C07H 21/04; C07D 265/38; C07K 16/46

(52) U.S. Cl. 435/6; 435/7.1; 530/391.1; 536/24.3; 544/103

(57) ABSTRACT

The invention concerns new oxazine derivatives of the general formula I

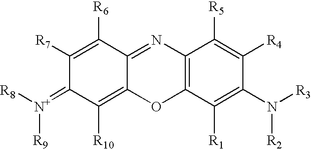

in which R₁, R₄, R₅, R₆, R₇, R₁₀ denote hydrogen, alkyl, hydroxy, halogen, carboxyl, sulfonyl or amino and

R₂, R₃

denote hydrogen, alkyl, alkoxy, polyoxyhydrocarbyl units, phenyl, phenylalkyl which can be substituted by hydroxy, sulfonyl, carboxy, amino, alkoxycarbonyl, in which R₂ with R₁ or R₃ with R₄ can form a saturated or unsaturated C2 or C3 bridge or R₂ with R₃ can form a saturated or unsaturated C4 or C5 bridge and

R₈, R₉

denote hydrogen, alkyl, alkoxy, polyoxyhydrocarbyl units, phenyl, phenylalkyl which can be substituted by hydroxy, sulfonyl, carboxy, amino, alkoxy-carbonyl in which R₈ with R₇ or R₉ with R₁₀ can form a saturated or unsaturated C2 or C3 bridge or R₈ with R₉ can form a saturated or unsaturated C4 or C5 bridge

and in which at least one of the residues R₂, R₃, R₈ or R₉ represents a non-bridge forming residue which is additionally substituted by an activated group capable of coupling or by a group that can be activated to couple.

and in which at least one of the residues R₂, R₃, R₈ or R₉ represents a bridge-forming residue which can be optionally substituted by alkyl.

The derivatives serve to produce fluorescent conjugates for use in immunoassays and for DNA analysis.

The invention concerns new oxazine dyes which are capable of coupling and their use as fluorescent markers in conjugates.

Markers or labels are necessary to carry out immunological assays and in DNA analytics which allow a quantification of the analyte after an analyte-specific reaction is completed.

Due to their high sensitivity fluorometric markers in particular have recently gained in importance. Thus the labelling of an antibody or a nucleotide with fluorescent dyes enables a direct quantification.

Widespread fluorescent dyes are for example FITC (fluorescein isothiocyanate), FLUOS (fluorescein N-hydroxysuccinimide ester), resorufin and rhodamine labels which, however, require relatively sophisticated light sources such as argon lasers for their excitation.

The rapid development of cheap laser diodes with an emission range of 630-780 nm which in addition are excellently suitable for constructing miniaturised systems, makes it desirable to have dyes which absorb in these wavelength regions.

Pentacyclic rhodamine dyes are described in EP-A-0 543 333 which can be used as a label. Their absorbance lies mainly in the range up to 660 nm.

In WO 88/047 77 phthalocyanine dyes are described which, however, have more than one functional group so that when they are conjugated for example with antibodies this leads to cross-linking and to product mixtures which require a very time-consuming purification.

Pentacyclic oxazine derivatives are described as laser dyes in U.S. Pat. No. 5,149,807. However, they do not have a functional group and are thus not suitable for specific coupling to biological molecules such as proteins, haptens and nucleic acids and such an application is not referred to.

A tricyclic activated oxazine compound from the Dojindo Company is known whose amine substituents are only substituted with non-ring forming alkyl groups. The corresponding conjugate has a very low quantum yield.

The object of the present invention is to provide dyes which are suitable for coupling to biological molecules which have a high quantum yield, absorb in an absorption range of 645-700 nm and have the lowest possible unspecific binding to biological compounds or to solid phases.

This object is achieved by the invention as characterized in the claims.

The invention concerns functional oxazine derivatives capable of coupling of the general formula I

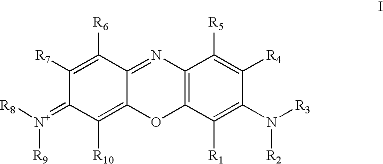

in which R₁, R₄, R₅, R₆, R₇, R₁₀ represent hydrogen, alkyl, alkoxy, hydroxy, halogen, carboxyl, sulfonyl or amino

and

R₂, R₃

denote hydrogen, alkyl, alkoxy, polyoxyhydrocarbyl units, phenyl, phenylalkyl which can be substituted by hydroxy, halogen, sulfonyl, carboxy, amino, alkoxycarbonyl, in which R₂ with R₁ or R₃ with R₄ can form a saturated or unsaturated C2 or C3 bridge or R₂ with R₃ can form a saturated or unsaturated C4 or C5 bridge and

R₈, R₉ denote hydrogen, alkyl, alkoxy, polyoxyhydrocarbyl units, phenyl, phenylalkyl which can be substituted by hydroxy, halogen, sulfonyl, carboxy, amino, alkoxycarbonyl in which R₈ with R₇ or R₉ with R₁₀ can form a saturated or unsaturated C2 or C3 bridge or R₈ with R₉ can form a saturated or unsaturated C4 or C5 bridge

and in which at least one of the residues R₂, R₃, R₈ or R₉ represents a non-bridge forming residue which is substituted by an activated group capable of coupling or by a group that can be activated to couple.

and in which at least one of the residues R₂, R₃, R₈ or R₉ represents a bridge-forming residue which optionally can be substituted by alkyl.

It is preferred when R₃ with R₄ and/or R₇ with R₈ forms a saturated or unsaturated C3 bridge.

It is particularly preferred when R₃ with R₄ and/or R₇ with R₈ form a C3 bridge while R₂ and/or R₉ represent non-bridge forming substituents, preferably alkyl and at least one non-bridge forming substituent is substituted with an activated group capable of coupling or with a group that can be activated.

The term “polyoxyhydrocarbyl units” is understood within the sense of the present invention as polymeric or oligomeric organic residues which are linked together by O bridges. In particular this term is understood as polyethers, polyoles, soluble carbohydrates, derivatives thereof or water-soluble polymers. Polyethyleneoxy groups are particularly preferred whose size is such that the molecular weight of the total compound is 800-1200, preferably about 1000. The aforementioned polyethyleneoxy groups improve the solubility, reduce the unspecific binding of the compounds to proteins and prevent a dimerization.

An alkyl group has 1-10 preferably 1-7 carbon atoms and can be branched or straight-chained; it especially preferably has 1 to 4 carbon atoms and is in particular for example methyl, ethyl, propyl, iso-propyl, butyl, iso-butyl or tert. butyl.

A phenylalkyl group with preferably 1-3 carbon atoms in the alkyl group is in particular a phenethyl or benzyl group.

Halogen is understood as fluorine, chlorine, bromine or iodine and preferably chlorine.

An alkoxy group in an alkoxycarbonyl group has 1-10, preferably 1-4 and especially preferably 1 or 2 C atoms.

In the compounds according to the invention of formula I at least one of the residues R₂, R₃, R₈, R₉ is preferably present as a non-bridge forming residue which is substituted with an activated group capable of coupling or with a group which can be activated for coupling. Such an activated group is in particular derived from an activatable carboxylic acid or sulfonic acid group and is for example an acid ester, an acid anhydride, an acid halogenide, preferably bromide in particular chloride or an N-hydroxysuccinimide ester. A linker compound such as DADOO may be inserted between the activated group and the non-bridge forming residue.

The table gives some examples for activated groups capable of coupling. A person skilled in the art knows further such groups from the synthetic chemistry for conjugates.

TABLE 1

| Activated group | Linkage with | Product |
|---|---|---|
| NHS ester | amines | amide |
| isothiocyanate | amines | thiourea |
| mixed anhydride | amines | amide |
| maleimide | thiol | thioether |
| thiol | maleimide | thioethers |
| haloacetyl | thiol | thioethers |
| hydrazine | aldehyde | hydrazones |
| amines | aldehyde | amine (after |
| | | reduction) |
| amines | reactive | amides |
| | carboxylic acid | |

Any anion suitable for charge neutralization and compatible with the cationic backbone can be used as the counterion; perchlorate is preferably used or the counterion is derived from a carboxyl or sulfonic group of one of the residues. In addition to the selection and combination of the residues, the selection of a suitable counterion enables the desired degree of lipophilicity to be optimized according to the intended application purpose.

Examples of particularly preferred substituents in the meaning of R₂, R₃, R₈ or R₉ are: hydrogen, methyl, carboxymethyl, ethyl, carboxyethyl, 3-sulfopropyl, 4-sulfobutyl, 3-carboxypropyl, 4-carboxy-butyl, 3-methoxycarbonylpropyl, 3-ethoxycarbonylpropyl, methoxyethoxyethyl, hydroxyethoxyethyl, benzyl.

When used as hydrophilic narkers it can be expedient to use asymmetric substituted products in which the residues R₂, R₃ are different from R₈, R₉ and for example denote a 3-carboxypropyl or 4-carboxybutyl group (R₂ or/and R₃) and a 3-sulfopropyl or 4-sulfobutyl group (R₈ and/or R₉).

Particularly preferred residues in compounds of formula I are:

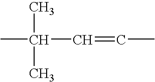

R₁, R₄, R₅, R₆ and/or R₁₀=hydrogen

R₃ with R₄ and/or R₈ with R₇=(CH₂)₃,

R₁ with R₂ or R₉ with R₁₀=(CH₂)₃

R₂, R₃, R₈, R₉=—CH₂—CH₃, —CH₂—CH₂—CH₂—COOH

R₂ with R₃ or R₈ with R₉=(CH₂)₄

The invention in addition concerns biologically active substances coupled to the fluorescent dyes according to the invention (conjugates) of formula II.

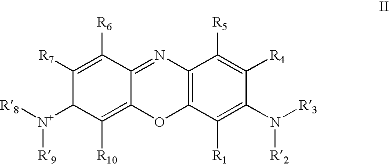

in which R₁, R₄, R₅, R₆, R₇, R₁₀ have the meaning stated above.

R₂′, R₃′

denote hydrogen, alkyl, alkoxy, polyoxyhydrocarbyl units, phenyl, phenylalkyl which can be substituted by hydroxy, sulfonyl, carboxy, amino, alkoxycarbonyl in which R₂′ with R₁ or R₃′ with R₄ can form a saturated or unsaturated C4 or C5 bridge and

R₈′, R₉′

denote hydrogen, alkyl, alkoxy, polyoxyhydrocarbyl units, phenyl, phenylalkyl which can be substituted by hydroxy, sulfonyl, carboxy, amino, alkoxycarbonyl in which R₂′ with R₁ or R₃′ with R₄ can form a saturated or unsaturated C4 or C5 bridge and in which at least one of the residues R₂′, R₃′, R₈′ or R₉′ represents a non-bridge forming residue which is coupled to a biologically active substance and in which at least one of the residues R₂′, R₃′, R₈′ and R₉′ represents a non-bridge forming residue which is optionally substituted by alkyl.

A biologically active substance is in particular understood as a hapten, antigen, antibody or fragment thereof, protein or mononucleotide or polynucleotide (PNA, RNA or DNA molecule).

The compounds for formula I can be obtained by condensation of 1,3-aminophenols of formula III with nitrosoaminophenols of formula IV.

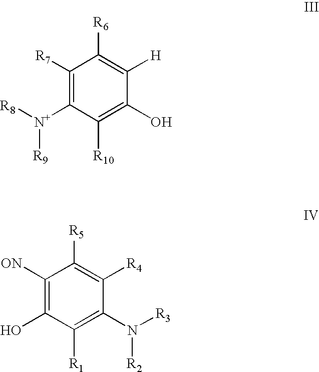

Activatable groups are activated to form groups capable of coupling according to known methods and coupled to reactive groups of biologically active molecules to form conjugates of formula II. In this process it is also possible to incorporate linkers between the activated groups and the biologically active molecules.

The compounds according to the invention provide new compounds which due to their spectroscopic properties (absorption maximum in the range between 645 to 700 nm) are very well suited for absorption dyes capable of coupling, in particular fluorescent dyes for application in hapten/and antibody-protein conjugates, for polynucleotide labelling and for dyeing latices (fluorescent latices). The quantum yield is high and is between 40 and 70% in ethanolic solution.

For application in hapten/antibody/protein or polynucleotide conjugates it is advantageous when the dyes are readily water-soluble. For this application compounds of the general formula I are preferably used in which R₂, R₃, R₈, R₉ are as hydrophilic as possible. These compounds are preferably asymmetrically substituted products which for example contain carboxyl as well as sulfonic acid groups. Coupling to the conjugate is achieved via one of these activated substituents of the residues R₂, R₃, R₈ or R₉ and in particular via a hydroxysuccinimide group.

Conjugates of the fluorescent dyes with haptens such as for example theophylline, digoxin, T₃, T₄ or protein such as for example antibodies are for example suitable for use in diagnostic systems in particular for fluorescence immunoassays.

A further subject matter of the present invention is a method for the determination of a first immunologically bindable substance which is characterized in that a conjugate of a compound according to the invention is used with a second immunologically bindable substance which can be the same or different from the first substance and that the change in absorbance or fluorescence or fluorescence polarization of the compound according to the invention caused by an immunological binding reaction which is specific for the first substance is determined as a measure for the amount of substance to be determined contained in the sample.

A further subject matter of the invention is the use of the conjugates according to the invention for immunoassays.

The compounds capable of coupling of formula I according to the invention are also suitable for the production of conjugates with mononucleotides or polynucleotides or PNA.

The invention therefore also concerns the use of these conjugates for DNA analysis.

### EXAMPLE 1

Synthesis of I

0.6 g (2.3 mmol) γ-(7-hydroxy-1,2,3,4-tetrahydroquinol-1-yl)butyric acid ethyl ester and 0.5 g (2,4 mmol) N-ethyl-7-hydroxy-6-nitroso-1,2,3,4-tetrahydroguinoline are boiled for 2 hours under reflux in 12 ml ethanol after addition of 2 ml 2.5 M hydrochloric acid. The solution is evaporated to dryness on a rotary evaporator. The residue is taken up in ethanol and chromatographically pre-purified over aluminium oxide. The ethyl ester of the target dye obtained in this manner has an absorption maximum in ethanol at 653 nm.

The ethyl ester is boiled for 30 min under reflux in a mixture of 30 ml acetone, 20 ml water and 1 ml 2.5 M hydrochloric acid. For purification the dye is chromatographed over silica gel (mobile solvent: firstly acetone-chloroform 3:1, then acetone and finally ethanol). 0.5 g I is obtained.

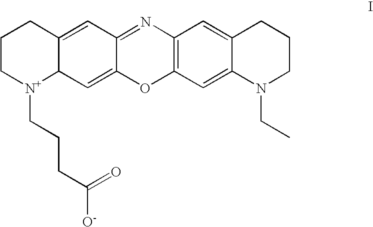

### EXAMPLE 2

Oxazine-N-hydroxysuccinimide ester II

40 mg oxazine I is dissolved together with 10 mg N-hydroxysuccinimide ester and 19 mg dicyclohexyl carbodiimide in 20 ml acetonitrile. It is allowed to stir for 4 hours at room temperature and the product mixture is rotary evaporated. It is purified by means of reverse phase silica gel.

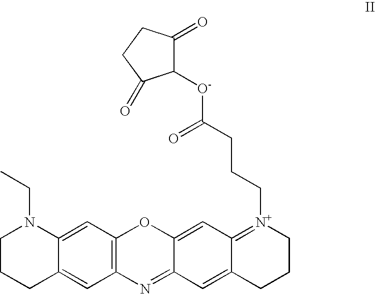

Digoxin-3-carboxymethyl ether-diaminodioxooctane conjugate III (Dig-CME-DADOO)

The hapten-fluorescent conjugate is obtained by reacting 11 mg I and 17.5 mg Dig-DADOO for 18 hours at room temperature in acetonitrile. The mixture is rotary evaporated and subsequently purified over silica gel, eluant chloroform-methanol-acetic acid 3:1:0:1.

Yield: 4 mg

Analysis: MS corresponds

Labelling of proteins with II

10 mg protein e.g. MAB<TSH>is dissolved in 1 ml sodium hydrogen phosphate buffer pH 8. A solution of a 10-fold molar excess of II dissolved in 500 μl DMSO is added to this. The reaction solution is shaken for 1 hour at room temperature. The conjugate is purified over a Sephadex G 50 column, mobile solvent buffer, dialysed three times against water and lyophilized.

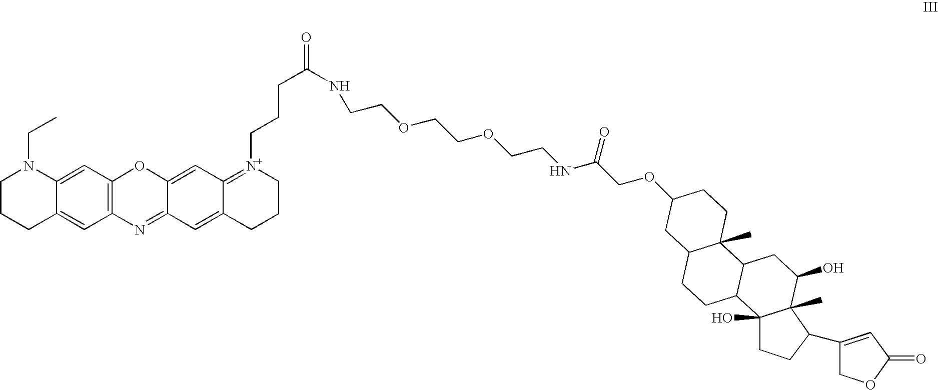

Synthesis of oxazines of formulae IV-X with activatable COOH groups

3 mmol substituted m-aminophenol or m-aminoanisol and 33 mmol 6-nitroso-3-amino-phenol are dissolved in a mixture of 20 ml ethanol and 1 ml 2.5 N hydrochloric acid and heated to reflux. The dye formation that takes place in this process is monitored spectrometrically (λmax in a range of 650-700 nm). The reaction is terminated when the dye concentration no longer increases.

The solution obtained in this way is evaporated to a volume of ca. 10 ml and then added dropwise to 200 ml 10% aqueous NaBF₄ solution. The dye tetrafluoroborate precipitates completely in this process. After decanting the supernatant liquid and filtering, the residue is taken up in 100 ml dichloromethane and this solution is washed 3 tines with 100 ml water each time. The solution is dried over Na₂SO₄ and rotary evaporated. In this process the dye accumulates as a viscous, almost black oil. Yield: 40-60%.

Hydrolysis:

The crude dye (ethyl ester) is dissolved in a mixture of 30 ml acetone, 15 ml water and 1 ml 2.5 N hydrochloric acid and heated to reflux. The hydrolysis is monitored by thin layer chromatography (silica gel, MeOH/H₂O 3:1). After almost complete reaction, the reaction solution is rotary evaporated at ca. 30° C. and the residue is purified by chromatography.

| | Reaction | Duration of | | |
|---|---|---|---|---|
| | time | hydrolysis | Purification | λmax. EtOH |
| IV | 1.5 h | 7 h | — | 660 |
| V | 70 min | 9 h | silica gel MS = | 650 |
| | | | chloroform/EtOH | |
| VI | 75 min | 9 h | silica gel MS = | 649 |
| | | | chloroform/EtOH | |
| VII | 40 min | 10 h | silica gel MS = | 672 |
| | | | chloroform/EtOH | |
| VIII | 90 min | 48 h | silica gel MS = | 682 |
| | | | chloroform/EtOH | |
| IX | 3 h | 24 h | silica gel MS = | 673 |
| | | | chloroform/EtOH | |
| X | 60 min | 24 h | silica gel MS = | 672 |
| | | | chloroform/EtOH | |

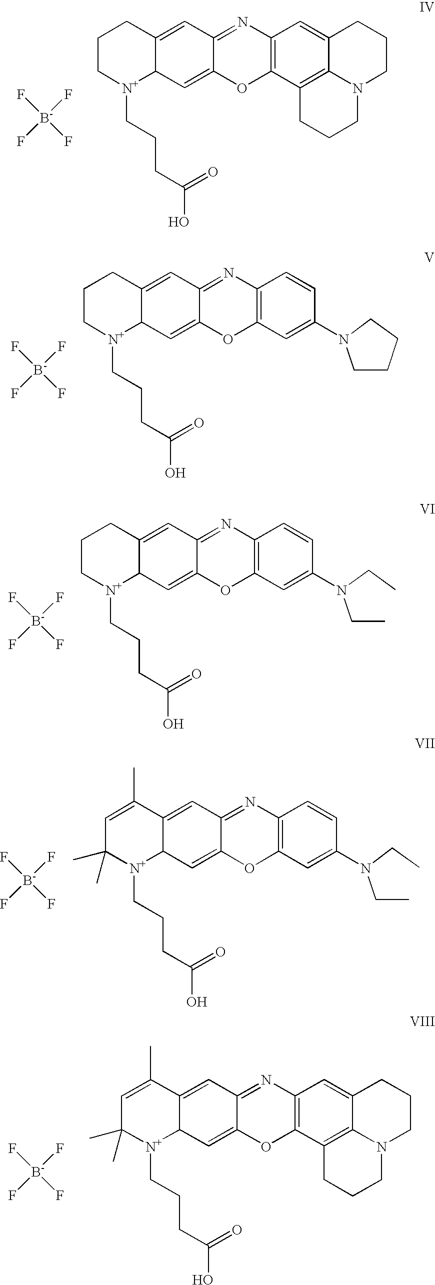

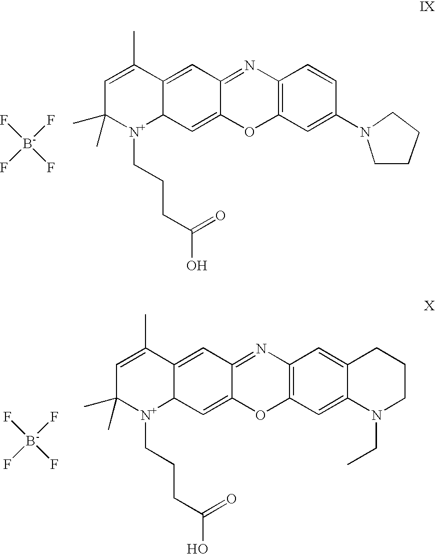

1. A compound of formula: 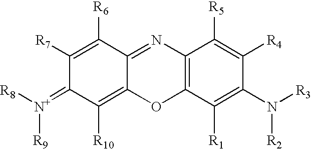
wherein R₁, R₄, R_{6,} R₇, and R₁₀ are each hydrogen, an alkyl of from 1 to 10 carbon atoms, an alkoxyl of 1 to 10 carbon atoms, hydroxy, halogen, carboxyl, sulfonyl or amino;
R₂ and R₃ are each hydrogen, an alkyl of 1 to 10 carbon atoms, an alkoxy of 1 to 10 carbon atoms, polyoxyhydrocarbyl, phenyl or phenylalkyl, R₂ and R₃ optionally substituted by a substituent selected from the group consisting of hydroxy, sulfonyl,: carboxyl, amino, and alkoxycarbonyl, wherein R₂ and R₁ or R₃ and R₄ can form a saturated or unsaturated C2 or C3 bridge or R₂ and R₃ can form a saturated or unsaturated C4 or C5 bridge;
R₈, and R₉ are each hydrogen, an alkyl of 1 to 10 carbon atoms, an alkoxy of 1 to 10 carbon atoms, polyoxhydrocarbyl, phenyl or phenylalkyl, R₈ and R₉ optionally substituted by a substituent selected from the group consisting of hydroxy, sulfonyl, carboxyl, amino, alkoxycarbonyl wherein R₈ and R₇ or R₉ and R₁₀ can form a saturated or unsaturated C2 or C3 bridge, or R₈ and R₉ can form a saturated or unsaturated C4 or C5 bridge;
wherein at least one of R₂, R₃, R₈ and R₉ is a non-bridge forming residue which is substituted by an activated group or by a group that can be activated to couple; and
at least one of R₂, R₃, R₈ and R₉ is a bridge-forming residue.

2. The compound of claim 1, wherein each of R₁ thru R₁₀ are alkyl of 1 to 7 carbon atoms.

3. The compound according to claim 1, wherein:
said R₁ thru R₁₀ are each alkyl of 1 to 4 carbon atoms;
said R₁ thru R₁₀ are each alkoxy of 1 to 4 carbon atoms;
said R₂ and R₃ are each phenyl alkyl, wherein said alkyl is 1 to 3 carbon atoms; and
R₈ and R₉ are each phenylalkyl, wherein said alkyl is 1 to 3 carbon atoms.

4. The compound according to claim 3, wherein said phenylalkyl is phenethyl or benzyl.

5. The compound according to claim 1, wherein said R₁ thru R₁₀ is alkoxy of 1 to 2 carbon atoms.

6. The compound according to claim 1, wherein said R₃ with R₄ or said R₇ with R₈ form a saturated or unsaturated C3 bridge.

7. The compound according to claim 6, wherein one of said R₂ or R₉ are each a non-bridge forming substituent of which at least one is substituted by an activated group or by a group that can be activated to couple.

8. The compound according to claim 7, wherein the non-bridge forming substituent is alkyl.

9. The compound according to claim 1, wherein said polyoxyhydrocarbyl is a polyethyleneoxy group whose size is such that the molecular weight of the total compound is from about 800 to about 1200.

10. The compound according to claim 7, wherein the molecular weight of said total compound is about 1000.

11. The compound according to claim 1, wherein said activated group is an acid ester, an acid anhydride, or an acid halide.

12. The compound according to claim 11, wherein the halide of said acid halide is one of bromide or chloride.

13. The compound according to claim 11, wherein said acid ester is N-hydroxysuccinimide ester.

14. The compound of claim 1 further comprising a linker compound inserted between said activated group and said non-bridge forming residue.

15. A conjugate of formula: 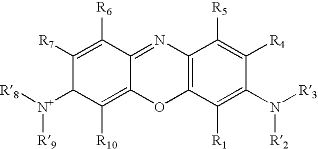
wherein R₁, R₄, R₅, R₆, R₇, R₁₀ are each hydrogen, an alkyl of from 1 to 10 carbon atoms, alkoxyl of 1 to 10 carbon atoms, hydroxy, halogen, carboxyl, sulfonyl or amino;
polyoxyhydrocarbyl, phenyl or phenylalkyl, R₂′ and R₃′ optionally substituted by a substituent selected from the group consisting of hydroxy, sulfonyl, carboxyl, amino, and alkoxycarbonyl, wherein R₂′ and R₁ or R₃′ and R₄ can form a saturated or unsaturated C2 or C3 bridge or R₂′ and R₃′ can form a saturated or unsaturated C4 or C5 bridge;
R₈′ and R₉′ are each hydrogen, an alkyl of 1 to 10 carbon atoms, an alkoxy of 1 to 10 carbon atoms, polyoxhydrocarbyl, phenyl or phenylalkyl, R₈′ and R₉′ optionally or substituted by substituents selected from the group consisting of hydroxy, sulfonyl, carboxyl, amino, alkoxycarbonyl wherein R₈′ and R₇ or R₉′ and R₁₀ can form a saturated or unsaturated C2 or C3 bridge, or R₈′ and R₉′ can form a saturated or unsaturated 4 to 5 carbons bridge;
wherein at least one of R₂′, R₃′, R₈′ and R₉′ is a non-bridge forming residue which is substituted with a biologically active group; and
at least one of R₂′, R₃′, R₈′ and R₉′ is a bridge-forming residue.

16. The conjugate of claim 15, wherein R₃′ with R₄′ or R₇′ with R₈′ forms a saturated or unsaturated C3 bridge.

17. The conjugate of claim 16, wherein one of R₂′ or R₉′ are each a non-bridge forming substituent of which at least one is substituted by a biologically active group.

18. The conjugate of claim 17, wherein said biologically active group is one of a hapten, antigen, antibody or protein.

19. The conjugate of claim 17, wherein said biologically active group is a mononucleotide or polynucleotide.

20. The conjugate of claim 15, wherein said bridge-forming residue of said at least one of R₂′, R₃′, R₈′ and R₉′ is substituted with by an alkyl of 1-10 carbon atoms.

21. A method for detecting a first immunologically bindable substance in a sample, comprising:
contacting said sample with the conjugate of claim 15, and a second immunologically bindable substance; and
determining any change in absorbance or florescence or fluorescence polarization of said conjugate, wherein said change is indicative of said first immunologically bindable substance in said sample.
wherein said change is indicative of said first immunologically bindable substance in said sample.

22. The method of claim 21, wherein said biologically active group is selected from the group consisting of a hapten, antigen, antibody, and a protein.

23. The method of claim 21, wherein said biologically active group is one of a mononucleotide and a polynucleotide.

24. The method of claim 21, wherein said method is an immunoassay.

25. The method of claim 21, wherein said method is a nucleic acid determining assay.

26. A process for producing the compound of claim 1 comprising:
reacting a first compound of formula 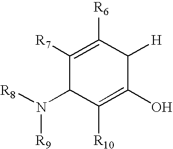
with a second compound of formula 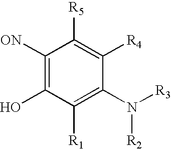
wherein R₁, R₄, R₅, R₆, R₇, and R₁₀ are each hydrogen, an alkyl of from 1 to 10 carbon atoms, an alkoxyl of 1 to 10 carbon atoms, hydroxy, halogen, carboxyl, sulfonyl or amino;
R₂ and R₃ are each hydrogen, an alkyl of 1 to 10 carbon atoms, an alkoxy of 1 to 10 carbon atoms, polyoxyhydrocarbyl, phenyl or phenylalkyl, R₂ and R₃ optionally substituted by a substituent selected from the group consisting of hydroxy, sulfonyl, carboxyl, amino, and alkoxycarbonyl, wherein R₂ and R₁ or R₃ and R₄ can form a saturated or unsaturated C2 or C3 bridge or R₂ and R₃ can form a saturated or unsaturated C4 or C5 bridge;
R₈ and R₉ are each hydrogen, an alkyl of 1 to 10 carbon atoms, an alkoxy of 1 to 10 carbon atoms, polyoxhydrocarbyl, phenyl or phenylalkyl, R₈ and R₉ optionally substituted by a substituent selected from the group consisting of hydroxy, sulfonyl, carboxyl, amino, alkoxycarbonyl wherein R₈ and R₇ or R₉ and R₁₀ can form a saturated or unsaturated C2 or C3 bridge, or R₈ and R₉ can form a saturated or unsaturated C4 or C5 bridge;
wherein at least one of R₂, R₃, R₈ and R₉ is a non-bridge forming residue which is substituted by an activated group or by a group that can be activated to couple; and
at least one of R₂, R₃, R₈ and R₉ is a bridge-forming residue, under conditions favorable to form said compound.

27. The process of claim 26, wherein said first compound is a 1,3-aminophenol and said second compound is a nitrosoaminophenol.
